(19) United States
(12) Patent Application Publication
(10) Pub. No.: US 2026/0321426 A1
(43) Pub. Date: Oct. 15, 2026

(54) Growth Assessment for Pulmonary Nodules

(71) Applicant: Icahn School of Medicine at Mount Sinai, New York, NY (US)

(72) Inventors: David Yankelevitz, New York, NY (US); Rowena Yip, New York, NY (US); Claudia I. Henschke, New York, NY (US)

(73) Assignee: Icahn School of Medicine at Mount Sinai, New York, NY (US)

(21) Appl. No.: 19/107,505

(22) PCT Filed: Aug. 30, 2023
(86) PCT No.: PCT/US2023/031555

§ 371 Date: Feb. 27, 2025

Related U.S. Application Data
(60) Provisional application No. 63/402,437, filed on Aug. 30, 2022.

(51) Int. Cl. G16H 50/30; G16B 40/00; G16B 45/00; G16H 10/40

(52) U.S. Cl. CPC G16H 50/30; G16B 40/00; G16B 45/00; G16H 10/40

(57) ABSTRACT

A diagnostic application retrieves, from a database, data describing a size of nodules associated with lung cancer in individuals and a change in the size of the nodules after a plurality of time periods between scans of the nodules. The diagnostic application determines lung-cancer cure rates for the individuals with lung cancer based on the plurality of time periods between scans of the nodules. The diagnostic application receives patient information associated with a patient that identifies that the patient has a patient nodule that is associated with lung cancer. The diagnostic application whether the patient nodule is a fast-growing cancer based on the patient information. Responsive to the nodule being the fast-growing cancer, the diagnostic application prioritizes a scheduling of a diagnostic workup for the patient.

### CROSS REFERENCE TO RELATED APPLICATIONS

This application a § 371 national stage of PCT International Application No. PCT/US23/31555, filed Aug. 30, 2023, claiming the benefit of U.S. Provisional Application Ser. No. 63/402,437 filed on Aug. 30, 2022. The disclosure of these prior applications are considered part of and are incorporated by reference in the disclosure of this application.

### FIELD

Growth assessment for pulmonary nodules is an important diagnostic tool, however, the impact on prognosis due to time delay for follow-up diagnostic scans needs to be considered.

### BACKGROUND

The pulmonary nodule is one of the most common imaging abnormalities with estimates of over 1.5 million new nodules identified each year in the United States. The frequency of pulmonary nodules will likely increase due to increasing utilization of computerized tomography (CT) scanning and penetration of CT screening for lung cancer, the leading cause of cancer death. As some of these nodules may be early lung cancers, which are highly curable, there is urgency to identify the cancerous ones before they progress.

Virtually all management protocols for lung nodules are based on the nodule size (volume or average diameter) and consistency (solid, part-solid, nonsolid). Focusing on solid cancers, usually the most aggressive ones, protocols provide three main options for workup based on size: 1) large solid nodules, as the probability of malignancy is so high, immediate intervention is needed; 2) small solid nodules, as the probability of malignancy is sufficiently low, they can safely be followed annually or even ignored; and 3) nodules whose size is in between the above two categories, management focuses on gaining additional information about the probability of malignancy. This can be obtained in a variety of ways with some protocols including well-defined options for positron emission topography (PET)-CT and minimally invasive biopsy procedures; however, the dominant approach to gain this information is to estimate whether the nodule is growing at a malignant rate. Protocols differ as to size thresholds and follow-up times for this in-between category. Decision theory analysis has found that the primary limitation of this approach is the hazard of a potentially curable lesion metastasizing during the follow-up delay. However, empirical data for making optimal determinations for its frequency is lacking, and protocols are primarily determined by consensus opinions.

The optimal time interval for obtaining a follow-up diagnostic scan is challenging as it also depends on nodule measurement accuracy. The greater the measurement accuracy, the shorter the follow-up time to confidently measure growth. Conversely, if there is likely no harm in waiting, a longer follow-up time would be better. Thus, a balance between accuracy of growth measurement and decrease in prognosis is needed.

The background description provided herein is for the purpose of presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Embodiments relate generally to a computer-implemented method that includes retrieving, from a database, data describing a size of nodules associated with lung cancer in individuals and a change in the size of the nodules after a plurality of time periods between scans of the nodules. The method further includes determining lung-cancer cure rates for the individuals with lung cancer based on the plurality of time periods between scans of the nodules. The method further includes receiving patient information associated with a patient that identifies that the patient has a patient nodule that is associated with lung cancer. The method further includes determining whether the patient nodule is a fast-growing cancer based on the patient information. The method further includes responsive to determining that the nodule is the fast-growing cancer, prioritizing a scheduling of a diagnostic workup for the patient.

In some embodiments, determining the lung-cancer cure rates includes determining a decrease in a lung-cancer cure rate when the diagnostic workup was delayed by 90, 180, and 365 days between an initial scan and a subsequent scan for fast-growing non-small-cell-lung cancer (NSCLC), moderate-growing NSCLC, and slow-growing NSCLC, wherein scheduling the diagnostic workup is based on determining the decrease in the lung-cancer cure rates. In some embodiments, determining the decrease in the lung-cancer cure rates includes determining lung-cancer-specific Kaplan-Meier (K-M) survival curves for the fast-growing NSCLC, the moderate-growing NSCLC, and the slow-growing NSCLC. In some embodiments, the method further includes plotting lung-cancer specific K-M survival rates for different tumor diameter categories. In some embodiments, the method further includes generating a linear regression model for the lung-cancer K-M survival rates for the individuals based on average initial tumor diameters.

In some embodiments, the method further includes estimating, using a fitted regression equation, an absolute change in the lung-cancer cure rates and a relative change in the lung-cancer cure rates for different initial tumor diameters and different delays in the diagnostic workup for fast-growing non-small-cell-lung cancer (NSCLC), medium-growing NSCLC, and slow-growing NSCLC. In some embodiments, the method further includes performing sensitivity analysis by fitting a linear regression model to combinations of the lung-cancer cure rates and average initial tumor diameters. In some embodiments, the patient information is received responsive to sampling blood of a patient to identify a blood-based marker and determining that the blood-based marker indicates that the patient has the patient nodule. In some embodiments, the patient information includes a computerized tomography (CT) scan that identifies the patient nodule.

According to one aspect, a system includes a processor and a memory coupled to the processor, with instructions stored thereon that, when executed by the processor, cause the processor to perform operations comprising: retrieving, from a database, data describing a size of nodules associated with lung cancer in individuals and a change in the size of the nodules after a plurality of time periods between scans of the nodules; determining lung-cancer cure rates for the individuals with lung cancer based on the plurality of time periods between scans of the nodules; receiving patient information associated with a patient that identifies that the patient has a patient nodule that is associated with lung cancer; determining whether the patient nodule is a fast-growing cancer based on the patient information; and responsive to determining that the patient nodule is the fast-growing cancer, prioritizing a scheduling of a diagnostic workup for the patient.

In some embodiments, determining the lung-cancer cure rates includes determining a decrease in a lung-cancer cure rate when the diagnostic workup was delayed by 90, 180, and 365 days between an initial scan and a subsequent scan for fast-growing non-small-cell-lung cancer (NSCLC), moderate-growing NSCLC, and slow-growing NSCLC, wherein scheduling the diagnostic workup is based on determining the decrease in the lung-cancer cure rates. In some embodiments, determining the lung-cancer cure rates includes determining lung-cancer-specific K-M survival curves for the fast-growing NSCLC, the moderate-growing NSCLC, and the slow-growing NSCLC. In some embodiments, the operations further include plotting lung-cancer specific K-M survival rates for different tumor diameter categories. In some embodiments, the operations further include generating a linear regression model for the lung-cancer K-M survival rates for the individuals based on average initial tumor diameters.

According to one aspect, non-transitory computer-readable medium with instructions that, when executed by one or more processors at a user device, cause the one or more processors to perform operations, the operations comprising: retrieving, from a database, data describing a size of nodules associated with lung cancer in individuals and a change in the size of the nodules after a plurality of time periods between scans of the nodules; determining lung-cancer cure rates for the individuals with lung cancer based on the plurality of time periods between scans of the nodules; receiving patient information associated with a patient that identifies that the patient has a patient nodule that is associated with lung cancer; determining whether the patient nodule is a fast-growing cancer based on the patient information; and responsive to determining that the patient nodule is the fast-growing cancer, prioritizing a scheduling of a diagnostic workup for the patient.

In some embodiments, determining the lung-cancer cure rates includes determining a decrease in a lung-cancer cure rate when the diagnostic workup was delayed by 90, 180, and 365 days between an initial scan and a subsequent scan for fast-growing non-small-cell-lung cancer (NSCLC), moderate-growing NSCLC, and slow-growing NSCLC, wherein scheduling the diagnostic workup is based on determining the decrease in the lung-cancer cure rates. In some embodiments, determining the lung-cancer cure rates includes determining lung-cancer-specific K-M survival curves for the fast-growing NSCLC, the moderate-growing NSCLC, and the slow-growing NSCLC. In some embodiments, the operations further include plotting lung-cancer specific K-M survival rates for different tumor diameter categories. In some embodiments, the operations further include generating a linear regression model for the lung-cancer K-M survival rates for the individuals based on average initial tumor diameters. In some embodiments, the operations further include estimating, using a fitted regression equation, an absolute change in the lung-cancer cure rates and a relative change in the lung-cancer cure rates for different initial tumor diameters and different delays in the diagnostic workup for fast-growing NSCLC, medium-growing NSCLC, and slow-growing NSCLC.

### BRIEF DESCRIPTION OF THE DRAWINGS

The disclosure is illustrated by way of example, and not by way of limitation, in the accompanying drawings in which like reference numerals are used to refer to similar elements.

FIG. 1 is a block diagram of an example network environment, according to some embodiments described herein.

FIG. 2 is a block diagram of an example computing device, according to some embodiments described herein.

FIG. 3 illustrates a flow diagram of study participants, according to some embodiments described herein.

FIG. 4 illustrates lung cancer-specific K-M curves for individuals diagnosed with solid lung cancers 33 mm or less in average diameter without clinical evidence of lymph node or distant metastases identifying as a result of the baseline round of screening in the International Early Lung cancer Action Program (I-ELCAP) database, according to some embodiments described herein.

FIG. 5 illustrates K-M 10-year lung cancer-specific survival rates by baseline solid NSCLCs in I-ELCAP by average diameter in 5.0 mm increments, according to some embodiments described herein.

FIG. 6 illustrates estimated 10-year lung cancer cure rates by average tumor diameter for the baseline solid non-small-cell lung cancers in the I-ELCAP database, according to some embodiments described herein.

FIG. 7 illustrates a linear regression model of 10-year lung cancer-specific survival on tumor average diameter and maximum dimension of tumor, according to some embodiments described herein.

FIG. 8A illustrates absolute and relative change in estimated 10-year lung cancer cure rates by average tumor diameter among baseline solid cancers for a slow-growing tumor, according to some embodiments described herein.

FIG. 8B illustrates absolute and relative change in estimated 10-year lung cancer cure rates by average tumor diameter among baseline solid cancers for a moderate-growing tumor, according to some embodiments described herein.

FIG. 8C illustrates absolute and relative change in estimated 10-year lung cancer cure rates by average tumor diameter among baseline solid cancers for a fast-growing tumor, according to some embodiments described herein.

FIG. 9A illustrates a three-dimensional surface plot of the relationship between tumor diameter and time delay and the estimated 10-year lung cancer-specific cure rate by average tumor diameter for a fast-growing tumor, according to some embodiments described herein.

FIG. 9B illustrates a three-dimensional surface plot of the relationship between tumor diameter and time delay and the estimated 10-year lung cancer-specific cure rate by average tumor diameter for a moderate-growing tumor, according to some embodiments described herein.

FIG. 9C illustrates a three-dimensional surface plot of the relationship between tumor diameter and time delay and the estimated 10-year lung cancer-specific cure rate by average tumor diameter for a slow-growing tumor, according to some embodiments described herein.

FIG. 10 illustrates a flow diagram of an example method to schedule a diagnostic workup, according to some embodiments described herein.

### DETAILED DESCRIPTION

In some embodiments, the technology includes a system and method to estimate the decrease in prognosis that occurs while waiting to assess growth as part of the diagnostic process. The diagnostic process may include identification of nodules on baseline CT screening, as the intervals for follow-up scans are longer than those for newly identified nodules on subsequent annual rounds of screening. Also, the baseline round provides the paradigm for nodules incidentally seen on CT scans performed outside of the screening context. Beyond considerations regarding the screening paradigm, this type of information is essential when considering the benefit of anything influencing timing of treatment. This includes the performance of experimental treatment protocols such as neo-adjuvant window trials for Stage I lung cancers, which lead to delay in starting standard of care treatment. Along those same lines, there is enormous interest in developing new blood-based biomarkers for evaluating the indeterminate nodule. This approach advantageously eliminates the need to wait to assess for growth and in essence represents the main potential benefit that these tests would provide. Currently there is no method for integrating how these changes resulting from delays in diagnosis impact overall utility.

Using the data between 2003 and 2019 from the International Early Lung cancer Action Program (I-ELCAP), a prospective cohort study, the diagnostic application, as described herein, determined the size specific 10-year Kaplan-Meier lung cancer survival rates as surrogates for cure rates. The diagnostic application determined the change in lung cancer diameter after delays of 90-, 180-, and 365-days using three representative lung cancer volume doubling times (VDTs) of 60 (fast), 120 (moderate), and 240 (slow). We then determined the decrease in the lung cancer cure rate resulting from time between CT scans to assess for growth during the diagnostic workup.

Using a regression model of the 10-year lung cancer survival rates on lung cancer diameter, the estimated lung cancer cure rate of a 4.0 mm lung cancer with fast (60-day) VDT is 96.0% (95% CI: 95.2%-96.7%) initially, but it would decrease to 94.3% (95% CI: 93.2%-95%), 92.0% (95% CI: 90.5%-93.4%) and 83.6% (95% CI: 80.6%-86.6%) after delays of 90, 180, and 365 days, respectively. A 20.0 mm lung cancer with the same VDTs has an initial lower lung cancer cure rate of 79.9% (95% CI: 76.2%-83.5%) initially, and decreases more rapidly to 71.5% (95% CI: 66.4%-76.7%), 59.8% (95% CI: 52.4%-67.1%) and 17.9% (95% CI: 3.0%-32.8%) after the same delays of 90, 180, and 365 days.

As a result, the diagnostic application, as described herein, determined that the time between scans required to measure growth of lung nodules impacts prognosis with the effect being greater for fast-growing and larger cancers. The diagnostic application quantifies the extent of change in prognosis to address efficiencies of different management protocols, especially those that use blood-based markers and do not require a second scan.

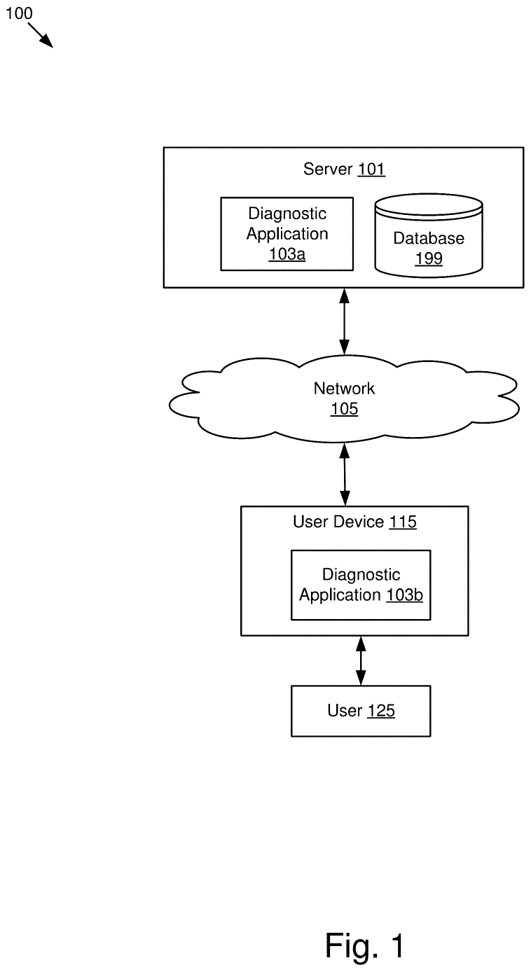

FIG. 1 illustrates a block diagram of an example environment 100. In some embodiments, the environment 100 includes a server 101 and user device 115, coupled via a network 105. User 125 may be associated with the user device 115. In some embodiments, the environment 100 may include other servers or devices not shown in FIG. 1. For example, the server 101 may include multiple servers 101 and the user device 115 may include multiple user devices 115a, n. In FIG. 1 and the remaining figures, a letter after a reference number, e.g., “115a,” represents a reference to the element having that particular reference number. A reference number in the text without a following letter, e.g., “115,” represents a general reference to embodiments of the element bearing that reference number.

The server 101 includes one or more servers that each include a processor, a memory, and network communication hardware. In some embodiments, the server 101 is a hardware server. The server 101 is communicatively coupled to the network 105. In some embodiments, the server 101 sends and receives data to and from the user device 115. The server 101 may include a diagnostic application 103 and a database 199.

In some embodiments, the diagnostic application 103a includes code and routines operable to retrieve, from a database, data describing a size of nodules associated with lung cancer in individuals and a change in the size of the nodules after a plurality of time periods between scans of the nodules. The diagnostic application 103a may determine lung-cancer cure rates for the individuals with lung cancer based on the plurality of time periods between scans of the nodules. For example, the plurality of time periods may include 90, 180, and 365 days of delay, where the delay is between an initial scan and a subsequent scan.

The diagnostic application 103a may receive patient information associated with a patient that identifies that the patient has a nodule that is associated with lung cancer. The patient information may include a computerized tomography (CT) scan, a blood sample where a blood-based marker was identified, etc. The diagnostic application 103a may determine whether the nodule is a fast-growing cancer. Responsive to determining that the nodule is the fast-growing cancer, the diagnostic application 103a may prioritize scheduling a diagnostic workup for the patient. For example, the diagnostic application 103a may prioritize a diagnostic delay of the patient above scheduling requests for other patients that have been diagnosed as having slow-growing cancers.

The database 199 may be a non-transitory computer readable memory (e.g., random access memory), a cache, a drive (e.g., a hard drive), a flash drive, a database system, or another type of component or device capable of storing data. The database 199 may also include multiple storage components (e.g., multiple drives or multiple databases) that may also span multiple computing devices (e.g., multiple server computers). The database 199 may store data associated with patients.

The user device 115 may be a computing device that includes a memory and a hardware processor. For example, the user device 115 may include a mobile device, a tablet computer, a desktop computer, a mobile telephone, a wearable device, a head-mounted display, a mobile email device, an augmented reality device, a virtual reality device, a reader device, or another electronic device capable of accessing a network 105.

The user device 115 includes a diagnostic application 103b. The diagnostic application 103b may perform the same functions as described above with reference to the diagnostic application 103a stored on the server 101.

In the illustrated embodiment, the entities of the environment 100 are communicatively coupled via a network 105. The network 105 may include a public network (e.g., the Internet), a private network (e.g., a local area network (LAN) or wide area network (WAN)), a wired network (e.g., Ethernet network), a wireless network (e.g., an 802.11 network, a Wi-Fi® network, or wireless LAN (WLAN)), a cellular network (e.g., a Long Term Evolution (LTE) network), routers, hubs, switches, server computers, or a combination thereof. Although FIG. 1 illustrates a single network 105 coupled to the server 101 and the user device 115, in practice one or more networks 105 may be coupled to these entities.

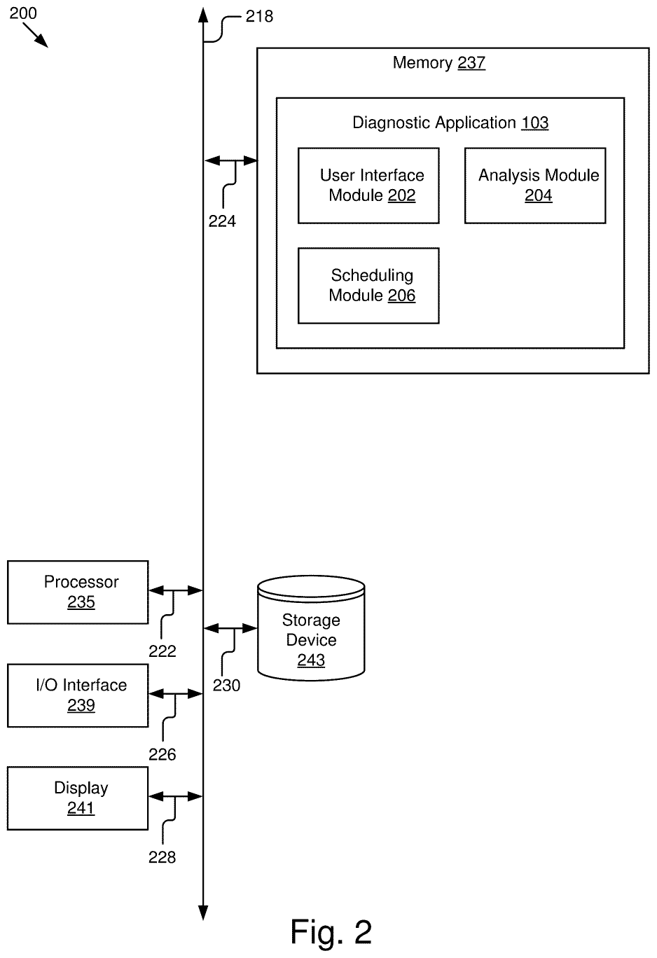

FIG. 2 is a block diagram of an example computing device 200 that may be used to implement one or more features described herein. Computing device 200 can be any suitable computer system, server, or other electronic or hardware device. In some embodiments, the computing device 200 is the user device 115. In some embodiments, the computing device 200 is the server 101.

In some embodiments, computing device 200 includes a processor 235, a memory 237, an Input/Output (I/O) interface 239, a display 241, and a storage device 243, all coupled via a bus 218. In some embodiments, the computing device 200 includes additional components not illustrated in FIG. 2. In some embodiments, the computing device 200 includes fewer components than are illustrated in FIG. 2. For example, in instances where the diagnostic application 103 is stored on the server 101 in FIG. 1, the computing device 200 may not include a display 241.

The processor 235 may be coupled to a bus 218 via signal line 222, the memory 237 may be coupled to the bus 218 via signal line 224, the I/O interface 239 may be coupled to the bus 218 via signal line 226, the display 241 may be coupled to the bus 218 via signal line 228, and the storage device 243 may be coupled to the bus 218 via signal line 230.

The processor 235 includes an arithmetic logic unit, a microprocessor, a general-purpose controller, or some other processor array to perform computations and provide instructions to a display device. Processor 235 processes data and may include various computing architectures including a complex instruction set computer (CISC) architecture, a reduced instruction set computer (RISC) architecture, or an architecture implementing a combination of instruction sets. In some implementations, the processor 235 may include special-purpose units, e.g., machine learning processor, audio/video encoding and decoding processor, etc. Although FIG. 2 illustrates a single processor 235, multiple processors 235 may be included. In different embodiments, processor 235 may be a single-core processor or a multicore processor. Other processors (e.g., graphics processing units), operating systems, sensors, displays, and/or physical configurations may be part of the computing device 200, such as a keyboard, mouse, etc.

The memory 237 stores instructions that may be executed by the processor 235 and/or data. The instructions may include code and/or routines for performing the techniques described herein. The memory 237 may be a dynamic random access memory (DRAM) device, a static RAM, or some other memory device. In some embodiments, the memory 237 also includes a non-volatile memory, such as a static random access memory (SRAM) device or flash memory, or similar permanent storage device and media including a hard disk drive, a compact disc read only memory (CD-ROM) device, a DVD-ROM device, a DVD-RAM device, a DVD-RW device, a flash memory device, or some other mass storage device for storing information on a more permanent basis. The memory 237 includes code and routines operable to execute the metaverse application 104, which is described in greater detail below.

I/O interface 239 can provide functions to enable interfacing the computing device 200 with other systems and devices. Interfaced devices can be included as part of the computing device 200 or can be separate and communicate with the computing device 200. For example, network communication devices, storage devices (e.g., memory 237 and/or storage device 247), and input/output devices can communicate via I/O interface 239. In another example, the I/O interface 239 can receive data from the server (or the user device depending on which diagnostic application is being used) and deliver the data to the diagnostic application 103 and components of the diagnostic application 103, such as the user interface module 202. In some embodiments, the I/O interface 239 can connect to interface devices such as input devices (keyboard, pointing device, touchscreen, microphone, sensors, etc.) and/or output devices (display 241, speakers, etc.).

Some examples of interfaced devices that can connect to I/O interface 239 can include a display 241 that can be used to display content, e.g., images, video, and/or a user interface of the metaverse as described herein, and to receive touch (or gesture) input from a user. Display 241 can include any suitable display device such as a liquid crystal display (LUNG CANCERD), light emitting diode (LED), or plasma display screen, cathode ray tube (CRT), television, monitor, touchscreen, three-dimensional display screen, a projector (e.g., a 3D projector), or other visual display device.

The storage device 243 stores data related to the diagnostic application 103. For example, the storage device 243 may store results from a query of the International Early Lung cancer Action Program (I-ELCAP) database, patient information, etc.

FIG. 2 illustrates a computing device 200 that executes an example diagnostic application 103 that includes a user interface module 202, an analysis module 204, and a scheduling module 206. In some embodiments, a single computing device 200 includes all the components illustrated in FIG. 2. In some embodiments, one or more of the components may be on different computing devices 200. For example, the user device may include the user interface module 202, while the analysis module 204 and the scheduling module 206 are implemented on the server. In some embodiments, different portions of one or more of modules 202, 204, 206 may be implemented on the user device and on the server.

The user interface module 202 generates graphical data for displaying a user interface for users associated with user devices. For example, the user interface may include information for running queries of the I-ELCAP database. In some embodiments, the user interface includes options for querying the I-ELCAP database for information with different factors. In some embodiments, the user interface displayed results for all participants diagnosed with non-small-cell-lung cancer (NSCLC) prompted by findings on the first, baseline round of low-dose CT (LDCT) screening who underwent surgical resection in the International Early Lung cancer Action Program (I-ELCAP) database between 2003 and 2019.

I-ELCAP research was conducted in accordance with the Declaration of Helsinki and was approved by the Western Institutional Review Board (IRB #20090325). All had signed informed consent in compliance with the Health Insurance Portability and Accountability Act and use of their data for these types of analyses was approved by the Institutional Review Board. Included are all clinical Stage I participants for whom the cancer manifested as a solid nodule that measured 30 mm or less in average diameter on the LDCT prior to date of surgery, regardless of when the diagnosis was made and who had no pre-treatment evidence of lymph node (N0) or distant (M0) metastases. Participants whose last pre-operative LDCT was more than six months before the date of surgery were excluded from the analysis.

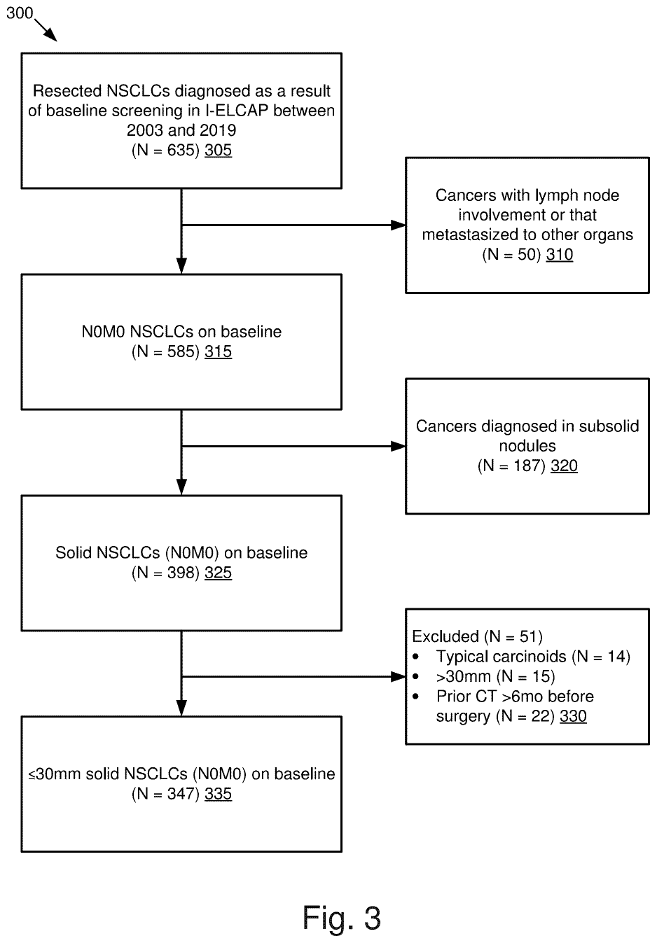

FIG. 3 illustrates a flow diagram 300 of study participants, according to some embodiments described herein. In this example, the first group (305) included 635 individuals with resected NSCLCs that were diagnosed as a result of baseline screening in I-ELCAP between 2003 and 2019. From the group of 635 individuals, 50 individuals were excluded (310) because they had cancers with lymph node involvement or cancers that metastasized to other organs. As a result, the remaining group (315) was reduced to 585 individuals with no pre-treatment evidence of lymph node (N0) or distant (M0) metastases.

Next, 187 individuals were excluded (320) where they had cancers diagnosed in subsolid nodules. As a result, the remaining group (325) was reduced to 398 individuals with solid NSCLCs on baseline with no pre-treatment evidence of lymph node (N0) or distant (M0) metastases.

Lastly, 51 individuals were excluded (330) that include 51 individuals that had typical carcinoids, 15 individuals with nodules greater than 33 mm, and 22 individuals that had a prior CT scan more than six months before surgery. As a result, the remaining group (335) included 347 individuals with solid NSCLCs less than or equal to 30 mm on baseline with no pre-treatment evidence of lymph node (N0) or distant (M0) metastases. Of the 347 individuals, the distribution by cell-type is given in Table 1 below.

TABLE 1
Distribution of lung cancer cell-types among clinical stage
IA non-small-cell solid lung cancers identified as a result
of the baseline round of screening in I-ELCAP databases (average
diameter of 30 mm or less on CT prior to diagnosis)

| Cell type | N (%) |
|---|---|
| Adenocarcinoma | 262 (75.5%) |
| Squamous cell | 56 (16.1%) |
| Large cell | 20 (5.8%) |
| Other | 9 (2.6%) |
| Total | 347 (100.0%) |

The median follow-up for these NSCLCs from time of diagnosis was 85.7 months (IQR: 41.8-148.0) with 42 lung cancer deaths over the study period.

The analysis module 204 determines the decrease in the lung cancer cure rate due to delays in starting definitive treatment that result in waiting for follow-up scans to assess nodules for growth. The analysis module 204 determined the size-specific 10-year Kaplan-Meier lung cancer survival rates as surrogates for cure rates as the asymptote of the survival curve has been reached at this point, and then estimated the decrease in the lung cancer cure rate when the diagnostic workup included a follow-up scan at 90, 180, and 365 days, separately for fast, moderate, and slow growing lung cancers, as detailed in the following steps.

The analysis module 204 calculated the increase of the average diameter of the solid NSCLC for three follow-up delays (90, 180, and 365 days) using three different tumor volume doubling times (VDTs), VDT of 60 days for fast (F)-growing NSCLCs, VDT of 120 days for moderate (M)-growing NSCLCs, and VDT of 240 days for slow (S)-growing NSCLCs as illustrated in Table 2 below.

TABLE 2
Change in average tumor diameter for fast (F: VDT =
60 days), moderate (M: VDT = 120 days), slow (S:
VDT = 240 days) according to the initial average tumor diameter.

| | Time between initial and follow-up CT | | | | | | | | |
|---|---|---|---|---|---|---|---|---|---|
| Initial | 90 days later | | | 180 days later | | | 365 days later | | |
| size (mm) | S | M | F | S | M | F | S | M | F |
| 4.0 | 4.4 | 4.8 | 5.7 | 4.8 | 5.7 | 8.0 | 5.7 | 8.1 | 16. |
| 5.0 | 5.5 | 5.9 | 7.1 | 5.9 | 7.1 | 10. | 7.1 | 10. | 20. |
| 6.0 | 6.5 | 7.1 | 8.5 | 7.1 | 8.5 | 12. | 8.5 | 12. | 24. |
| 7.0 | 7.6 | 8.3 | 9.9 | 8.3 | 9.9 | 14. | 9.9 | 14. | 28. |
| 8.0 | 8.7 | 9.5 | 11. | 9.5 | 11. | 16. | 11. | 16. | 32. |
| 9.0 | 9.8 | 10. | 12. | 10. | 12. | 18. | 12. | 18. | 36. |
| 10.0 | 10. | 11. | 14. | 11. | 14. | 20. | 14. | 20. | 40. |
| 11.0 | 12. | 13. | 15. | 13. | 15. | 22. | 15. | 22. | 44. |
| 12.0 | 13. | 14. | 17. | 14. | 17. | 24. | 17. | 24. | 48. |
| 13.0 | 14. | 15. | 18. | 15. | 18. | 26. | 18. | 26. | 53. |
| 14.0 | 15. | 16. | 19. | 16. | 19. | 28. | 19. | 28. | 57. |
| 15.0 | 16. | 17. | 21. | 17. | 21. | 30. | 21. | 30. | 61. |
| 16.0 | 17. | 19. | 22. | 19. | 22. | 32. | 22. | 32. | 65. |
| 17.0 | 18. | 20. | 24. | 20. | 24. | 34. | 24. | 34. | 69. |
| 18.0 | 19. | 21. | 25. | 21. | 25. | 36. | 25. | 36. | 73. |
| 19.0 | 20. | 22. | 26. | 22. | 26. | 38. | 27. | 38. | 77. |
| 20.0 | 21. | 23. | 28. | 23. | 28. | 40. | 28. | 40. | 81. |

Note.
VDT = Volume doubling time.
F = Fast.
M = Moderate.
S = Slow

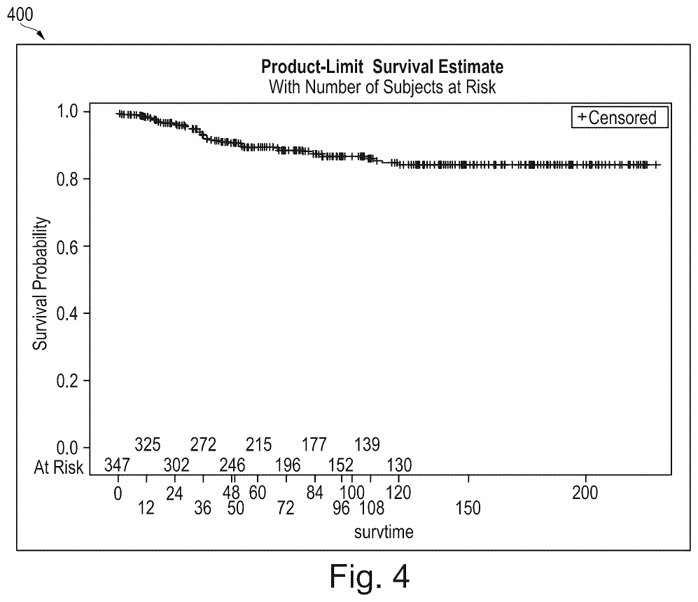

The analysis module 204 calculated the lung-cancer-specific survival for the 347 solid baseline NSCLCs using Kaplan-Meier method. FIG. 4 represents a graph 400 of the lung cancer specific Kaplan-Meier curves for individuals diagnosed with solid lung cancers 33 mm or less in average diameter without clinical evidence of lymph node or distant metastases identified as a result of the baseline round of screening in the I-ELCAP database.

Survival time was calculated from the date of surgery to the date of death, the date of last follow-up, or Dec. 31, 2019, whichever came first. The overall 10-year lung cancer survival rate was 84.1% (95% CI: 79.5%-88.7%).

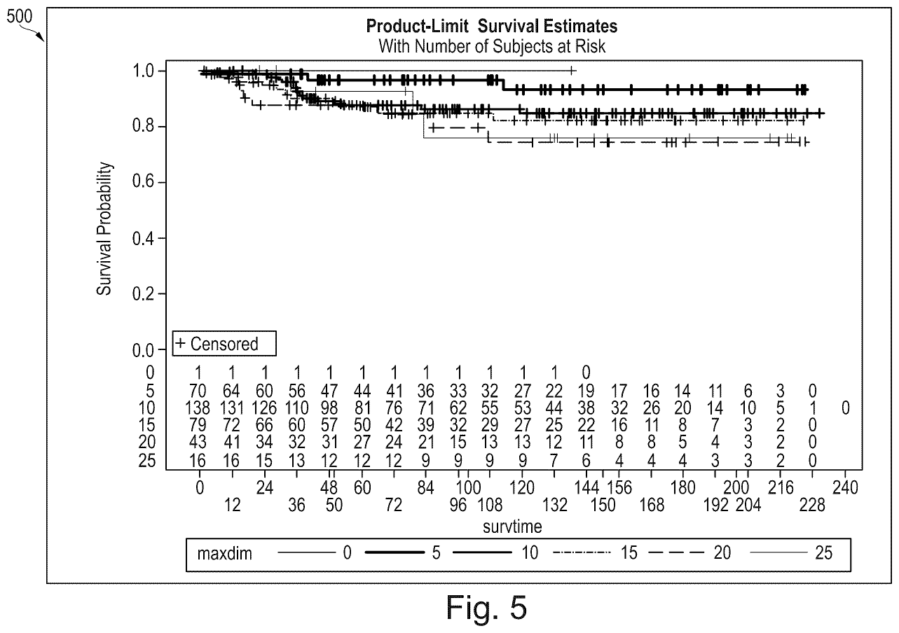

FIG. 5 illustrates a graph 500 of the Kaplan-Meier (K-M) 10-year lung cancer survival rates by average lung cancer diameter in 5.0 mm increments: ≤5.0 mm, 5.1-10.0 mm, 10.1-15.0 mm, 15.1-20.0 mm, 20.1-25.0 mm, and 25.1-30.0 mm. The average lung cancer diameter was the average of maximum tumor length and its perpendicular width, as it provides the best two-dimensional approximation of the tumor volume. For this reason, this threshold is used in both I-ELCAP and Lung-RADS protocols. As no patient with a NSCLC≤5.0 mm cancer died of lung cancer, the 10-year lung cancer survival was 100%. Ten-year lung cancer survival was 93.4% (95% CI: 85.7%-100.0%) for 5.1-10.0 mm lung cancers, 84.7% (95% CI: 77.7%-91.6%) for 10.1-15.0 mm lung cancers, 82.1% (95% CI: 72.0%-92.2%) for 15.1-20.0 mm lung cancer s, 74.4% (95% CI: 57.6%-91.1%) for 20.1-25.0 mm lung cancers, and 76.0% (95% CI: 52.1%-99.8%) for 25.1-30.0 mm lung cancers.

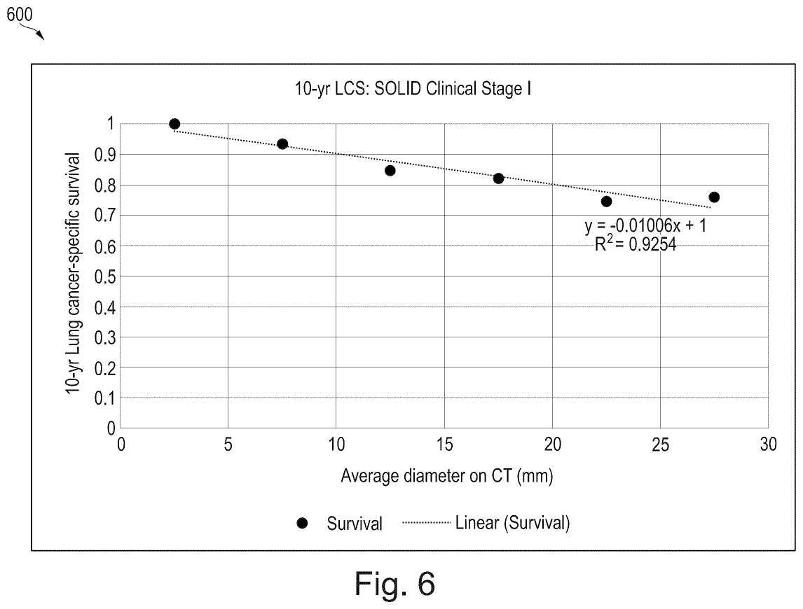

FIG. 6 illustrates a graph 600 of the estimated 10-year lung cancer cure rates by average tumor diameter for the baseline solid non-small-cell lung cancers in the I-ELCAP database. In FIG. 6, the vertical axis represents the 95% confidence interval of the mean 10-year lung cancers.

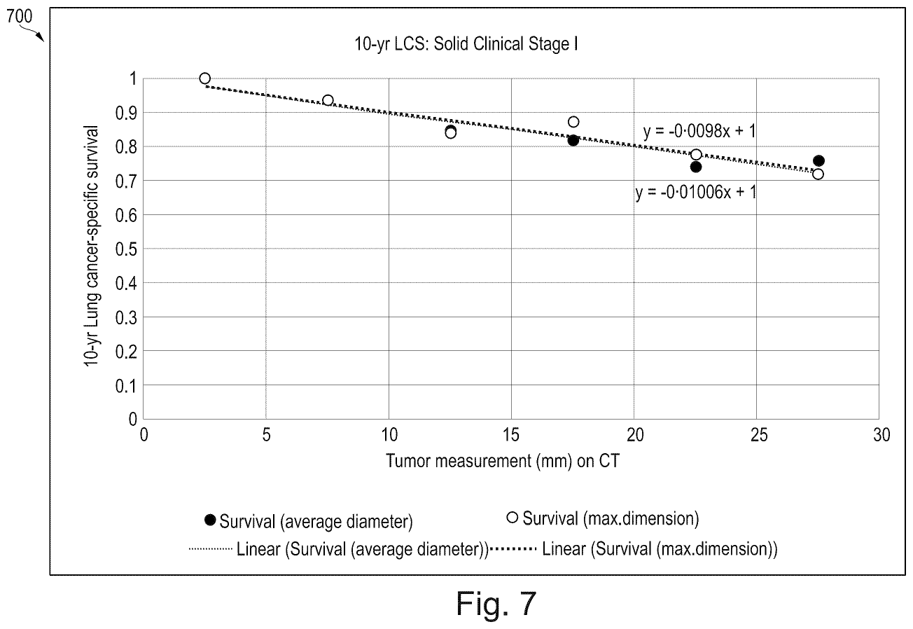

FIG. 7 illustrates a graph 700 of the estimated 10-year lung cancer-specific survival for the tumor average diameter and maximum dimension of the tumor.

The lung cancer cure rates (FIG. 6) by average diameters (FIG. 7) with the intercept for the regression model fixed at one (assumes 100% survival for individuals without NSCLCs) are shown.

The analysis module 204 used the fitted regression equation illustrated in FIG. 6 to estimate the absolute and relative change in the lung cancer cure rate for different initial diameters and three different delays (90, 180, and 365 days) in the diagnostic workup, separately for F-, M-, and S-growing lung cancers as defined in 1) above.

The analysis module 204 performed sensitivity analysis by sampling different 10-year lung cancer cure rates within the 95% confidence interval of each of the six tumor diameter categories as shown in FIG. 6. For this sensitivity analysis, the analysis module 204 fitted linear regression models to all possible combinations of cure rates and tumor diameters. Mean and standard deviation of the slopes from each of these models were computed and compared with the slope estimated from the initial regression model.

In some embodiments, the analysis module 204 quantified the decrease in the lung cancer cure rates due to delays that occur as part of the diagnostic workup for different initial lung cancer diameters (4.0 mm-20.0 mm), lung cancer aggressiveness (F-, M-, S-growing), using three different follow-up times (90, 180, and 365 days). Using the methodology described above, the analysis module 204 assessed changes in lung cancer cure rates for all nine combinations of lung cancer aggressiveness and follow-up times.

Table 2 provides the change in lung cancer diameter for different follow-up times for tumors with three different VDTs. A fast-growing (F) lung cancer with 4.0 mm in average diameter reaches a diameter of 5.7 mm, 8.0 mm, and 16.3 mm at follow-up times of 90, 180, and 365 days, respectively while a slow-growing(S) one would have an average diameter of 4.4 mm, 4.8 mm, and 5.7 mm respectively. This illustrates the impact of growth rate increases with increasing time. In addition, the initial size of the cancer is important. A fast-growing (F) lung cancer with 20.0 mm in average diameter reaches a diameter of 28.3 mm, 40.0 mm, and 81.6 mm (no longer Stage I based on size alone) for follow-up times of 90, 180, and 365 days respectively. The analysis module 204 determines that while the proportional change for the 4.0 mm and 20.0 mm lung cancers is identical for each follow-up interval, the absolute change in diameter is larger for lung cancer with larger initial diameter.

To estimate the change in lung cancer cure rates for different initial lung cancer diameters (4.0 to 20.0 mm), delays (90, 180, and 365 days) in follow-up, and tumor aggressiveness (F-, M-, and S-growing lung cancers), the analysis module 204 applied a fitted regression equation and its slope parameter estimate, (β)=−0.01006 (95% CI: −0.01189 to −0.00823) as illustrated in Table 3 below.

TABLE 3
Change in average tumor diameter for fast (F: VDT = 60 days), moderate
(M: VDT = 120 days), slow (S: VDT = 240 days) according to the
initial average diameter for the baseline non-small-cell lung cancer. Using
the estimated regression equation for 10-yr lung cancer survival rates (in
5-mm increments), the 10-yr lung cancer survival rate is given by the following
regression equation, lung cancer survival rate = (−0.01006*avg. diameter) + 1

| | 10-yr | Decrease(absolute) in estimated 10 y lung cancer survival rate | | | | | | | | |
|---|---|---|---|---|---|---|---|---|---|---|
| | LCS @ | due to tumor growth from initial average diameter | | | | | | | | |
| Initial | initial | | | | | | | | | |
| diameter | diameter | 90 days later | | | 180 days later | | | 365 days later | | |
| (mm) | (%) | S | M | F | S | M | F | S | M | F |
| 4.0 | 96.0 | 0.4 | 0.8 | 1.7 | 0.8 | 1.7 | 4.0 | 1.7 | 4.1 | 12.4 |
| 5.0 | 95.0 | 0.5 | 0.9 | 2.1 | 0.9 | 2.1 | 5.0 | 2.1 | 5.1 | 15.5 |
| 6.0 | 94.0 | 0.5 | 1.1 | 2.5 | 1.1 | 2.5 | 6.0 | 2.5 | 6.1 | 18.6 |
| 7.0 | 93.0 | 0.6 | 1.3 | 2.9 | 1.3 | 2.9 | 7.0 | 2.9 | 7.1 | 21.6 |
| 8.0 | 92.0 | 0.7 | 1.5 | 3.3 | 1.5 | 3.3 | 8.0 | 3.4 | 8.2 | 24.7 |
| 9.0 | 90.9 | 0.8 | 1.7 | 3.7 | 1.7 | 3.7 | 9.1 | 3.8 | 9.3 | 27.9 |
| 10.0 | 89.9 | 0.9 | 1.9 | 4.1 | 1.9 | 4.1 | 10.1 | 4.2 | 10.3 | 31.0 |
| 11.0 | 88.9 | 1.0 | 2.1 | 4.6 | 2.1 | 4.6 | 11.1 | 4.6 | 11.3 | 34.1 |
| 12.0 | 87.9 | 1.1 | 2.3 | 5.0 | 2.3 | 5.0 | 12.1 | 5.1 | 12.3 | 37.1 |
| 13.0 | 86.9 | 1.2 | 2.5 | 5.4 | 2.5 | 5.4 | 13.1 | 5.5 | 13.4 | 40.2 |
| 14.0 | 85.9 | 1.3 | 2.6 | 5.8 | 2.6 | 5.8 | 14.1 | 5.9 | 14.4 | 43.4 |
| 15.0 | 84.9 | 1.4 | 2.8 | 6.2 | 2.8 | 6.2 | 15.1 | 6.3 | 15.4 | 46.5 |
| 16.0 | 83.9 | 1.4 | 3.0 | 6.6 | 3.0 | 6.6 | 16.1 | 6.7 | 16.4 | 49.5 |
| 17.0 | 82.9 | 1.5 | 3.2 | 7.0 | 3.2 | 7.0 | 17.1 | 7.2 | 17.4 | 52.6 |
| 18.0 | 81.9 | 1.6 | 3.4 | 7.5 | 3.4 | 7.5 | 18.1 | 7.6 | 18.4 | 55.7 |
| 19.0 | 80.9 | 1.7 | 3.6 | 7.9 | 3.6 | 7.9 | 19.1 | 8.0 | 19.5 | 58.9 |
| 20.0 | 79.9 | 1.8 | 3.8 | 8.3 | 3.8 | 8.3 | 20.1 | 8.5 | 20.5 | 62.0 |

*Numbers (in red) were extrapolated from the 10-yr lung cancer equation developed using ≤30 mm nodules data. Use with caution.
Note.
VDT = Volume doubling time.
F = Fast.
M = Moderate.
S = Slow.
LCS = Lung cancer specific survival.
Yr = Year

The parameter estimate of the slope implies that for every 1 mm increase in average lung cancer diameter, 10-year lung cancer cure rates decrease by 1.0%. Sensitivity analysis of all the possible combinations of lung cancer cure rates by tumor diameter provided a mean parameter estimate (β) of −0.01023, suggesting that the coefficient is consistent for different cure rates within the 95% confidence interval. Using the maximum tumor diameter, the parameter estimate was β=−0.0098, slightly smaller than the mean parameter estimate obtained using the average diameter of the lung cancer, which was β=−0.01 (as illustrated in FIG. 7). As the difference is very small, the analysis module 204 used the parameter estimate for the change in lung cancer cure rate associated with a one-unit change in average lung cancer diameter in any further calculations of lung cancer cure rates.

Using the fitted linear regression of 10-yr lung cancer cure rate on average tumor diameter, the analysis module 204 estimated the absolute decrease in the 10-year lung cancer cure rate for average tumor diameters for lung cancers, ranging from 4.0 mm to 20.0 mm, separately for S-, M-, and F-growing NSCLCs. For example, a baseline solid NSCLC with an initial average diameter of 15.0 mm has an estimated 10-yr lung cancer cure rate of 84.9% (95% CI: 82.2%-87.7%). After a follow-up of 90 days, the lung cancer will reach an average diameter of 16.4 mm, 17.8 mm, and 21.2 mm for S-, M-, and F-growing lung cancers, respectively and the corresponding 10-year lung cancer cure rate for these lung cancers will be 83.5% (95% CI: 80.5%-86.5%), 82.1% (95% CI: 78.8%-85.4%) and 78.7% (95% CI: 74.8%-82.6%). These are absolute decreases in lung cancer cure rates of 1.4%, 2.8%, and 6.2%, respectively. Using upper and lower limits of the 95% confidence intervals, the decrease may be as high as 7.2% (87.7%-80.5%), 8.9% (87.7%-78.8%) and 12.9% (87.7%-74.8%).

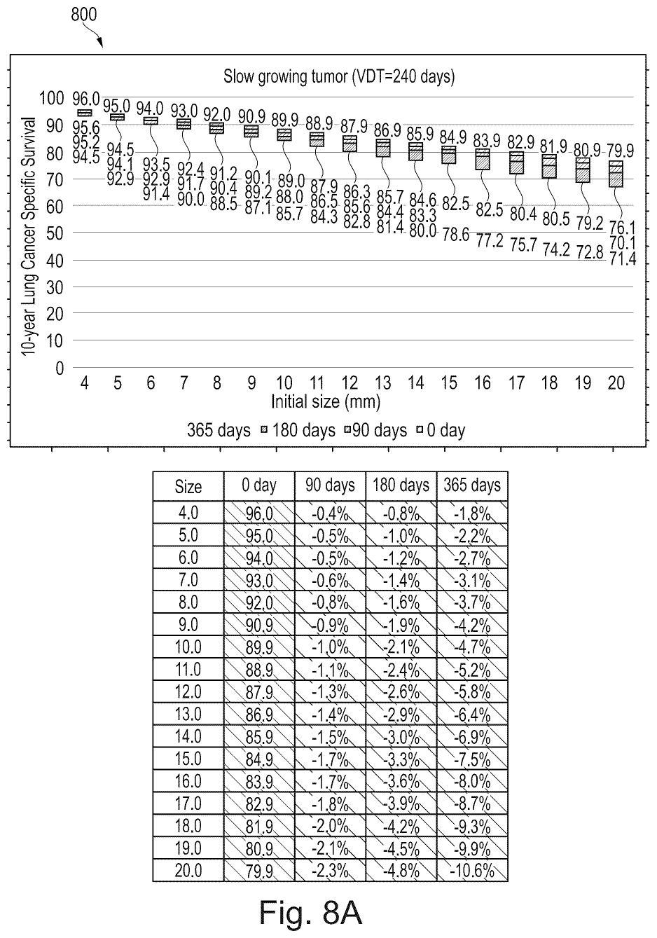

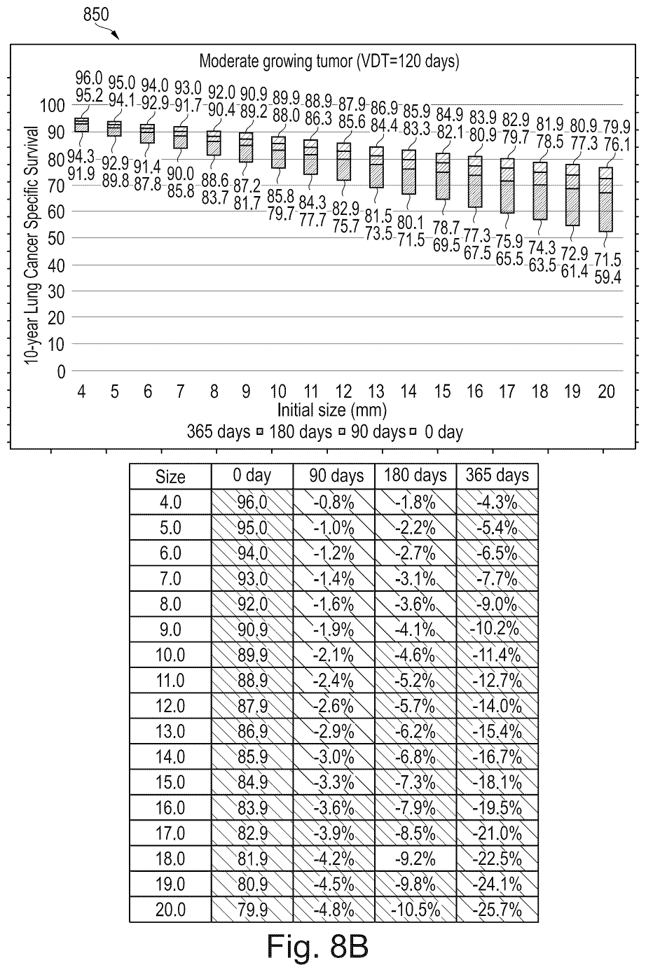

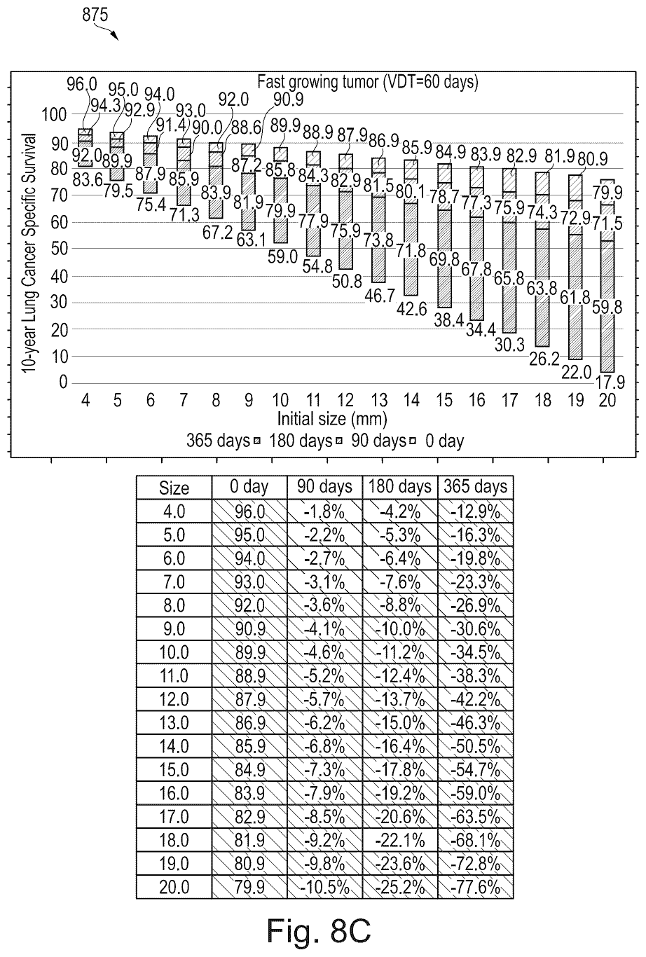

FIG. 8A includes a graph 800 of absolute and relative change in estimated 10-year lung cancer cure rates by average tumor diameter among baseline solid cancers for slow-growing cancers (VDT of 240 days). The stacked bar chart with each bar indicating the 10-year lung cancer cure rate for cancers with initial specified diameter and how the lung cancer cure rate changes with different time delays. The four numbers on each bar, from top to bottom, give the 1-year lung cancer cure rate given the initial diameter, then the absolute change in 1-=year lung cancer cure rate after a delay of 90-, 180-, and 365-days, respectively.

The table summarizes the initial 10-year lung cancer cure rate and the relative change in lung cancer cure rate by cancer diameter and follow-up delay of 90, 180, and 365 days. For example, for a 20-mm moderate-growing lung cancer (VDT of 120 days), the initial 10-year lung cancer cure rate is estimated to be 79.9%. The lung cancer cure rate decreases to 76.1% after 90 days, 71.5% after 180 days and 59.4% after 365 days, these correspond to a −4.8%, −10.5% and −25.7% relative decrease in 10-year lung cancer cure rates.

FIG. 8B includes a graph 850 of absolute and relative change in estimated 10-year lung cancer cure rates by average tumor diameter among baseline solid cancers for moderate-growing cancers (VDT of 120 days). FIG. 8C includes a graph 875 of absolute and relative change in estimated 10-year lung cancer cure rates by average tumor diameter among baseline solid cancers for fast-growing cancers (VDT of 60 days).

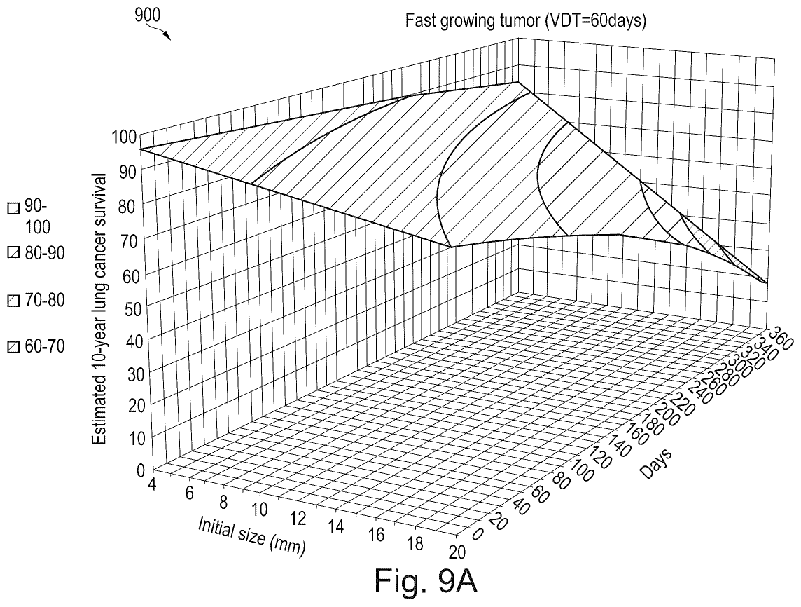

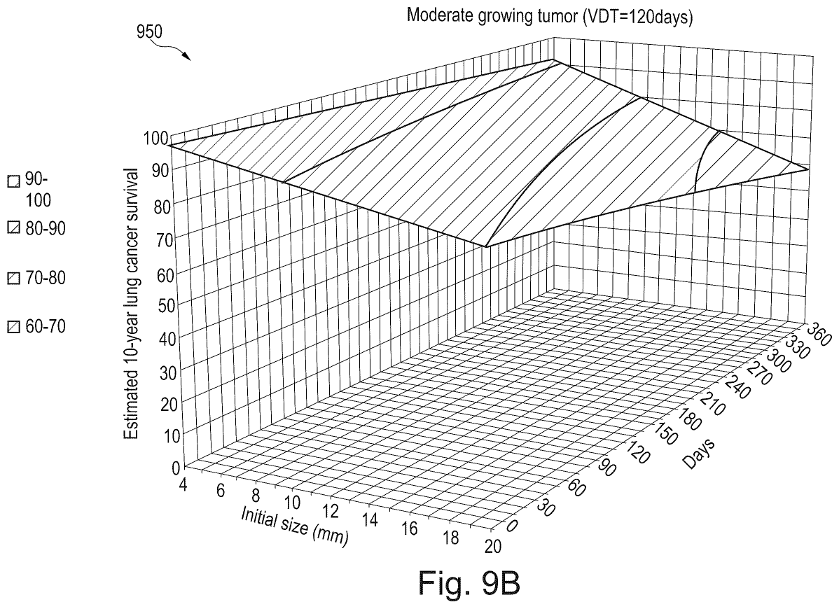

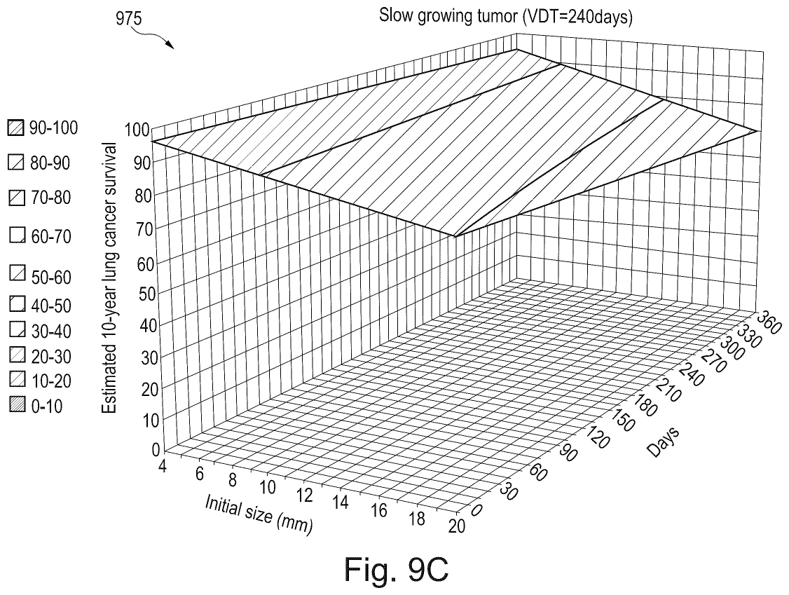

FIG. 9A illustrates a three-dimensional surface plot 900 of the relationship between tumor diameter and time delay and the estimated 10-year lung cancer-specific cure rate by average tumor diameter for a fast-growing tumor where the VDT is 60 days, according to some embodiments described herein. The three-dimensional surface plot 900 illustrates the relationship between tumor diameter and time delay on a continuous scale and the estimated 10-year lung cancer-specific cure rate. The X-axis represents the initial tumor diameter in millimeters, the y-axis represents the estimated 10-year lung cancer-specific cure rate, and the z-axis is the number of days up to one year. The shades of the plot 900 indicate ranges of 10-year lung cancer-specific cure rates.

For a fast-growing lung cancer identified on baseline screening, a 365-day delay of a follow-up CT for a small nodule would have a smaller impact on the 10-year lung cancer cure rate compared to a nodule of a larger size. For example, 365-day delay for the follow-up CT for a 4-mm fast growing lung cancer would decrease the 10-year LC cure rate from a range of 90-100% to one of 80-90%. However, the same 365-day delay for a 20-mm fast growing tumor would decrease the 10-year LC cure rate from 70-80% to 0-10%.

FIG. 9B illustrates a three-dimensional surface plot 950 of the relationship between tumor diameter and time delay and the estimated 10-year lung cancer-specific cure rate by average tumor diameter for a moderate-growing tumor where the VDT is 120 days, according to some embodiments described herein.

FIG. 9C illustrates a three-dimensional surface plot 975 of the relationship between tumor diameter and time delay and the estimated 10-year lung cancer-specific cure rate by average tumor diameter for a slow-growing tumor where the VTD is 240 days, according to some embodiments described herein.

Comparing a 4.0 mm lung cancer with a 20.0 mm lung cancer, both lung cancers having the same growth rate (60-day VDT) demonstrates the impact of initial cancer size. The 4.0 mm lung cancer would have an expected lung cancer cure rate of 96.0% (95% CI: 95.2%-96.7%) initially, and the cure rate would decrease to 94.3% (95% CI: 93.2%-95.3%), 92.0% (95% CI: 90.5%-93.4%) and 83.6% (95% CI: 80.6%-86.6%) after a delay of 90, 180, and 365 days, respectively. The 20.0 mm lung cancer, which starts at a lower lung cancer cure rate of 79.9% (95% CI: 76.2%-83.5%), decreases more rapidly to 71.5% (95% CI: 66.4%-76.7%), 59.8% (52.4%-67.1%) and 17.9% (95% CI: 3.0%-32.8%) after the same delay of 90, 180, and 365 days, respectively, a dramatic difference in lung cancer cure rates. While proportional change in diameter remains the same, the absolute change in diameter means that for larger lung cancers, the lung cancer cure rate declines more rapidly for any specified follow-up interval. The VDT (even when only considering its impact on change in the lung cancer diameter) also determines the change in prognosis, as it determines the absolute change in diameter over a specified follow-up interval. Slower VDTs will have less impact. Nevertheless, for any given doubling time, the absolute decrease in the lung cancer cure rate is greater for larger lung cancers.

Quantifying the decrease in the lung cancer cure rates due to delays inherent to the process of obtaining follow-up scans to assess growth in the diagnostic management algorithm is of critical importance in designing an efficient management protocol.

The analysis module 204 determined that for any given VDT, the proportional change in cancer diameter over time was size independent, but the absolute change was size dependent. This means that for larger lung cancers, the lung cancer cure rate declines more rapidly during a given follow-up interval. The lung cancer VDT (even when only considering its impact on change in size) also determines the decrease in lung cancer cure rate as it determines the absolute increase in tumor diameter over a specified diagnostic follow-up interval. Thus, slower VDTs have less impact on the lung cancer diameter for the same follow-up interval. Nevertheless, for any given VDT and diagnostic follow-up interval, the absolute decrease in the lung cancer cure rate is greater for larger lung cancers.

The scheduling module 206 schedules a diagnostic workup for patients. In some embodiments, the scheduling module 206 prioritizes a scheduling of a diagnostic workup for a patient when the patient where the analysis module 206 has predicted that the lung cancer is a fast-growing lung cancer. In some embodiments, the scheduling module 206 prioritizes scheduling of the diagnostic workup for larger cancers, such as cancers that are categorized as being Stage II cancers or lung cancers that are greater than 6.0 mm.

In some embodiments, prioritizing the scheduling of the diagnostic workup includes scheduling the patient before other patients that have less aggressive lung cancers. In some embodiments, the scheduling module 206 prioritizing the scheduling of the diagnostic workup based on a confidence score associated with a determination that the patient nodule is a fast-growing cancer. For example, the scheduling module 206 may schedule a diagnostic workup for a first patient with a higher confidence score than a second patient with a lower confidence score.

The choice of follow-up time is a balance between the decreased lung cancer prognosis and the time needed to overcome measurement error to assess growth. In other words, whether the measured change is genuine or just due to measurement error. The primary reason for the current choices of three- and six-month follow-up intervals are based primarily on the need to overcome measurement error. Likewise, a size threshold of 6 mm for the scheduling module 206 to initiate a workup of nodules on the baseline round of lung cancers prior to the next annual follow-up scan also reflects the difficulty in measuring true change for nodules smaller than this. For a given VDT and diagnostic follow-up interval, the capability to assess true growth will be better for larger nodules, but the decrease in prognosis, if diagnosed as lung cancer, will also be greater. This also implies that a shorter time interval for larger nodules will be sufficient to overcome the measurement error. This tends to be reflected in modern management protocols.

Based on the above considerations, it becomes clear that this balance between the ability to measure change in size versus change in prognosis is critical for designing management protocols also leads to critical new insights. For example, if it were possible to predict growth rates based on a nodule's morphologic features, then the scheduling module 206 using a 6.0 mm size threshold to initiate workup on baseline lung cancers prior to one year could be made shorter, since rapidly growing cancers below this size could overcome the threshold for measurement error in a shorter time interval. The potential gain in prognosis for this subset of nodules is balanced against the additional workups that would potentially be initiated.

Likewise, for larger nodules (≥6.0 mm), where the scheduling module 206 uses a protocol with a shorter interval for follow-up, typically at either three or six months, the analysis module 204 identifies patients with slow VDTs. Thus, the analysis module 204 avoids obtaining additional scans where growth may not be apparent. For example, when the analysis module 204 uses this prediction model and the scheduling module 206 raises the work-up threshold from 6.0 mm to 7.0 mm or even 8.0 mm, the frequency of positive results on baseline scanning decrease from a current rate of 10.2% on baseline (6 mm threshold) down to 7.1% or 5.1% with 7.0 mm or 8.0 mm thresholds. The downside for higher size thresholds is the scheduling module 206 may not schedule a diagnostic workup during the first year after baseline screening, which results in a small number of cancers (5-6% of cancers) growing for an entire year until growth is identified on the first annual repeat screening. However, if only slow-growing cancers have delayed diagnosis, the impact on prognosis would be minimized.

These considerations also provide for a direct estimate of the potential benefit of newer diagnostic tests that assess malignancy status without waiting to assess for growth. This includes blood-based biomarkers. Here, the potential benefit is that there is no time delay in the scheduling module 206 implementing the next steps from the protocol. Therefore, the balance is between the gain in prognosis of the new test versus additional potential false positive and false negatives compared to the traditional methodology of waiting to assess for growth.

In some embodiments, the analysis module 206 determines the prognosis for solid lung cancers based on size. While size is a critical factor for prognosis, there are other factors that affect prognosis, notably lung cancer biology, although its impact is partially reflected by lung cancer VDTs. In addition, it is generally accepted that faster growing lung cancers have worse prognosis including an increased propensity to metastasize. In some embodiments, the analysis module 206 determines estimated lung cancer cure rates based on the assumption that lung cancers would not develop additional lymph node or distant metastases during the follow-up interval, so the estimate for decrease in prognosis only considered increases in lung cancer diameter, and assumed the lung cancer remained in clinical Stage I. Therefore, the analysis module 206 generates estimates that represent a lower bound for the impact of delayed diagnosis. In addition, cancer size was taken from pre-operative CT within six months of surgery, thus the actual nodule size at the time of surgery would be larger resulting in an underestimation in our size-specific lung cancer cure rates. However, this bias may be systematic across all ≤30 mm Stage IA lung cancers, which on average minimizes its impact when calculating change in prognosis as the biases will be in the same direction for both size estimates. In some embodiments, the analysis module 206 determines estimates based on changes in diameter and not volume, which is becoming the preferred method for measuring change in nodule size. In some embodiments, the analysis module 206 determines estimates based on changes in volume.

In conclusion, delays in diagnosis associated with commonly used management protocols that allow for growth assessment cause predictable and quantifiable decreases in lung cancer prognosis. The decrease is more profound for faster-growing and larger lung cancers. This type of information, while essential in developing efficient management protocols, has previously been unavailable. It also provides quantitative information useful for providing insight into any diagnostic or therapeutic protocol that affects timing of treatment.

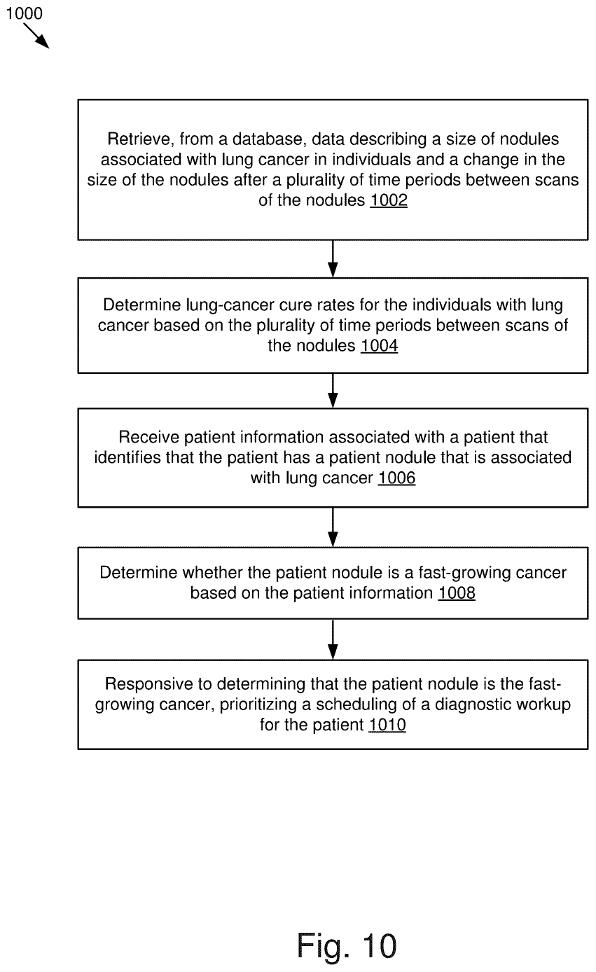

FIG. 10 illustrates a flow diagram of an example method 1000 to schedule a diagnostic workup. In some embodiments, all or portions of the method 1000 are performed by the diagnostic application 103 stored on the user device 115 or the server 101 of FIG. 1 and/or the diagnostic application 103 stored on the computing device 200 of FIG. 2.

The method may begin at block 1002. At block 1002, data is retrieved from a database describing a size of nodules associated with lung cancer in people and a change in the size of the nodules after a plurality of time periods between scans of the modules. In some embodiments, the database is the International Early Lung Cancer Action Program (I-ELCAP) database. Block 1002 may be followed by block 1004.

At block 1004, lung-cancer cure rates are determined for the individuals with lung cancer based on the plurality of time periods between scans of the nodules. In some embodiments, determining the lung-cancer cure rates includes determining a decrease in a lung-cancer cure rate when the diagnostic workup was delayed by 90, 180, and 365 days for fast-growing NSCLC, moderate-growing NSCLC, and slow-growing NSCLC, wherein scheduling the diagnostic workup is based on determining the decrease in the lung-cancer cure rates. In some embodiments, determining the lung-cancer cure rates includes determining lung-cancer-specific K-M survival curves for the fast-growing NSCLC, the moderate-growing NSCLC, and the slow-growing NSCLC.

In some embodiments, the method 1000 further includes plotting lung-cancer specific K-M survival rates for different tumor diameter categories. In some embodiments, the method 1000 further includes generating a linear regression model for the lung-cancer K-M survival rates for the individuals based on average initial tumor diameters.

In some embodiments, the method 1000 further includes estimating, using a fitted regression equation, an absolute change in the lung-cancer cure rates and a relative change in the lung-cancer cure rates for different initial tumor diameters and different delays in the diagnostic workup for fast-growing non-small-cell-lung cancer (NSCLC), medium-growing NSCLC, and slow-growing NSCLC. In some embodiments, the method 1000 further includes performing sensitivity analysis by fitting a linear regression model to combinations of the lung-cancer cure rates and average initial tumor diameters. Block 1004 may be followed by block 1006.

At block 1006, patient information that is associated with a patient is received that identifies that the patient has a patient nodule that is associated with lung cancer. In some embodiments, the patient information is received responsive to sampling blood of a patient to identify a blood-based marker and determining that the blood-based marker indicates that the patient has the nodule. In some embodiments, the patient information includes a CT scan, X-ray, magnetic imagining resonance (MRI), a bone scan, a positron emission tomography (PET) scan, thoracentesis, bronchoscopy, biopsy, etc. that identifies the patient nodule. Block 1006 may be followed by block 1008.

At block 1008, it is determined whether the nodule is a fast-growing cancer based on the patient information. For example, the blood-based marker may indicate that the patient nodule is a fast-growing cancer. In another example, one or more CT scans may indicate that the patient nodule is a fast growing cancer based on a growth of the patient nodule between scans, based on an identification of the appearance of the patient nodule in a particular time frame, etc. Block 1008 may be followed by block 1010.

At block 1010, responsive to determining that the nodule is the fast-growing cancer, a scheduling of a diagnostic workup for the patient is prioritized. In some embodiments, prioritizing the scheduling of the diagnostic workup is based on a probability that the patient nodule is a fast-growing cancer. For example, a patient with a 90% change of having a fast-growing cancer is scheduled for an earlier diagnostic workup than a patient with a 50% chance of having a fast-growing cancer.

The methods, blocks, and/or operations described herein can be performed in a different order than shown or described, and/or performed simultaneously (partially or completely) with other blocks or operations, where appropriate. Some blocks or operations can be performed for one portion of data and later performed again, e.g., for another portion of data. Not all of the described blocks and operations need be performed in various embodiments. In some embodiments, blocks and operations can be performed multiple times, in a different order, and/or at different times in the methods.

Various embodiments described herein include obtaining data from various sensors in a physical environment, analyzing such data, generating recommendations, and providing user interfaces. Data collection is performed only with specific user permission and in compliance with applicable regulations. The data are stored in compliance with applicable regulations, including anonymizing or otherwise modifying data to protect user privacy. Users are provided clear information about data collection, storage, and use, and are provided options to select the types of data that may be collected, stored, and utilized.

In the above description, for purposes of explanation, numerous specific details are set forth in order to provide a thorough understanding of the specification. It will be apparent, however, to one skilled in the art that the disclosure can be practiced without these specific details. In some instances, structures and devices are shown in block diagram form in order to avoid obscuring the description. For example, the embodiments can be described above primarily with reference to user interfaces and particular hardware. However, the embodiments can apply to any type of computing device that can receive data and commands, and any peripheral devices providing services.

Reference in the specification to “some embodiments” or “some instances” means that a particular feature, structure, or characteristic described in connection with the embodiments or instances can be included in at least one embodiments of the description. The appearances of the phrase “in some embodiments” in various places in the specification are not necessarily all referring to the same embodiments.

Some portions of the detailed descriptions above are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic data capable of being stored, transferred, combined, compared, and otherwise manipulated. It has proven convenient at times, principally for reasons of common usage, to refer to these data as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms including “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission, or display devices.

The embodiments of the specification can also relate to a processor for performing one or more steps of the methods described above. The processor may be a special-purpose processor selectively activated or reconfigured by a computer program stored in the computer. Such a computer program may be stored in a non-transitory computer-readable storage medium, including, but not limited to, any type of disk including optical disks, ROMS, CD-ROMs, magnetic disks, RAMS, EPROMs, EEPROMs, magnetic or optical cards, flash memories including USB keys with non-volatile memory, or any type of media suitable for storing electronic instructions, each coupled to a computer system bus.

The specification can take the form of some entirely hardware embodiments, some entirely software embodiments or some embodiments containing both hardware and software elements. In some embodiments, the specification is implemented in software, which includes, but is not limited to, firmware, resident software, microcode, etc.

Furthermore, the description can take the form of a computer program product accessible from a computer-usable or computer-readable medium providing program code for use by or in connection with a computer or any instruction execution system. For the purposes of this description, a computer-usable or computer-readable medium can be any apparatus that can contain, store, communicate, propagate, or transport the program for use by or in connection with the instruction execution system, apparatus, or device.

A data processing system suitable for storing or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

What is claimed is:

1. A computer-implemented method comprising:
retrieving, from a database, data describing a size of nodules associated with lung cancer in individuals and a change in the size of the nodules after a plurality of time periods between scans of the nodules;
determining lung-cancer cure rates for the individuals with lung cancer based on the plurality of time periods between scans of the nodules;
receiving patient information associated with a patient that identifies that the patient has a patient nodule that is associated with lung cancer;
determining whether the patient nodule is a fast-growing cancer based on the patient information; and
responsive to determining that the patient nodule is the fast-growing cancer, prioritizing a scheduling of a diagnostic workup for the patient.

2. The method of claim 1, wherein determining the lung-cancer cure rates includes determining a decrease in a lung-cancer cure rate when the diagnostic workup was delayed by 90, 180, and 365 days between an initial scan and a subsequent scan for fast-growing non-small-cell-lung cancer (NSCLC), moderate-growing NSCLC, and slow-growing NSCLC, wherein scheduling the diagnostic workup is based on determining the decrease in the lung-cancer cure rates.

3. The method of claim 2, wherein determining the decrease in the lung-cancer cure rates includes determining lung-cancer-specific Kaplan-Meier (K-M) survival curves for the fast-growing NSCLC, the moderate-growing NSCLC, and the slow-growing NSCLC.

4. The method of claim 3, further comprising plotting lung-cancer specific K-M survival rates for different tumor diameter categories.

5. The method of claim 4, further comprising generating a linear regression model for the lung-cancer K-M survival rates for the individuals based on average initial tumor diameters.

6. The method of claim 1, further comprising estimating, using a fitted regression equation, an absolute change in the lung-cancer cure rates and a relative change in the lung-cancer cure rates for different initial tumor diameters and different delays in the diagnostic workup for fast-growing non-small-cell-lung cancer (NSCLC), medium-growing NSCLC, and slow-growing NSCLC.

7. The method of claim 6, further comprising performing sensitivity analysis by fitting a linear regression model to combinations of the lung-cancer cure rates and average initial tumor diameters.

8. The method of claim 1, wherein the patient information is received responsive to sampling blood of a patient to identify a blood-based marker and determining that the blood-based marker indicates that the patient has the patient nodule.

9. The method of claim 1, wherein the patient information includes a computerized tomography (CT) scan that identifies the patient nodule.

10. A system comprising:
a processor; and
a memory coupled to the processor, with instructions stored thereon that, when executed by the processor, cause the processor to perform operations comprising:
retrieving, from a database, data describing a size of nodules associated with lung cancer in individuals and a change in the size of the nodules after a plurality of time periods between scans of the nodules;
determining lung-cancer cure rates for the individuals with lung cancer based on the plurality of time periods between scans of the nodules;
receiving patient information associated with a patient that identifies that the patient has a patient nodule that is associated with lung cancer;
determining whether the patient nodule is a fast-growing cancer based on the patient information; and
responsive to determining that the patient nodule is the fast-growing cancer, prioritizing a scheduling of a diagnostic workup for the patient.

11. The system of claim 10, wherein determining the lung-cancer cure rates includes determining a decrease in a lung-cancer cure rate when the diagnostic workup was delayed by 90, 180, and 365 days between an initial scan and a subsequent scan for fast-growing non-small-cell-lung cancer (NSCLC), moderate-growing NSCLC, and slow-growing NSCLC, wherein scheduling the diagnostic workup is based on determining the decrease in the lung-cancer cure rates.

12. The system of claim 11, wherein determining the decrease in the lung-cancer cure rates includes determining lung-cancer-specific Kaplan-Meier (K-M) survival curves for the fast-growing NSCLC, the moderate-growing NSCLC, and the slow-growing NSCLC.

13. The system of claim 12, wherein the operations further include plotting lung-cancer specific K-M survival rates for different tumor diameter categories.

14. The system of claim 13, wherein the operations further include generating a linear regression model for the lung-cancer K-M survival rates for the individuals based on average initial tumor diameters.

15. A non-transitory computer-readable medium with instructions that, when executed by one or more processors at a user device, cause the one or more processors to perform operations, the operations comprising:
retrieving, from a database, data describing a size of nodules associated with lung cancer in individuals and a change in the size of the nodules after a plurality of time periods between scans of the nodules;
determining lung-cancer cure rates for the individuals with lung cancer based on the plurality of time periods between scans of the nodules;
receiving patient information associated with a patient that identifies that the patient has a patient nodule that is associated with lung cancer;
determining whether the patient nodule is a fast-growing cancer based on the patient information; and
responsive to determining that the patient nodule is the fast-growing cancer, prioritizing a scheduling of a diagnostic workup for the patient.

16. The non-transitory computer-readable medium of claim 15, wherein determining the lung-cancer cure rates includes determining a decrease in a lung-cancer cure rate when the diagnostic workup was delayed by 90, 180, and 365 days between an initial scan and a subsequent scan for fast-growing non-small-cell-lung cancer (NSCLC), moderate-growing NSCLC, and slow-growing NSCLC, wherein scheduling the diagnostic workup is based on determining the decrease in the lung-cancer cure rates.

17. The non-transitory computer-readable medium of claim 16, wherein determining the decrease in the lung-cancer cure rates includes determining lung-cancer-specific Kaplan-Meier (K-M) survival curves for the fast-growing NSCLC, the moderate-growing NSCLC, and the slow-growing NSCLC.

18. The non-transitory computer-readable medium of claim 17, wherein the operations further include plotting lung-cancer specific K-M survival rates for different tumor diameter categories.

19. The non-transitory computer-readable medium of claim 18, wherein the operations further include generating a linear regression model for the lung-cancer K-M survival rates for the individuals based on average initial tumor diameters.

20. The non-transitory computer-readable medium of claim 15, wherein the operations further include estimating, using a fitted regression equation, an absolute change in the lung-cancer cure rates and a relative change in the lung-cancer cure rates for different initial tumor diameters and different delays in the diagnostic workup for fast-growing non-small-cell-lung cancer (NSCLC), medium-growing NSCLC, and slow-growing NSCLC.
